(19) United States
(12) Patent Application Publication
(10) Pub. No.: US 2021/0228515 A1
(43) Pub. Date: Jul. 29, 2021

(54) BENZOIC ACID OR A SALT AND DERIVATIVE THEREOF FOR USE IN PREVENTING OR TREATING ANTI-N-METHYL-D-ASPARTATE RECEPTOR ENCEPHALITIS

(71) Applicants: Ruu-Fen Tzang, Taipei (TW); Hsien-Yuan Lane, Taichung City (TW); EXCELSIOR PHARMATECH LABS, Taipei City (TW)

(72) Inventors: Ruu-Fen Tzang, Taipei (TW); Hsien-Yuan Lane, Taichung City (TW)

(21) Appl. No.: 17/258,180

(22) PCT Filed: Jul. 16, 2019
(86) PCT No.: PCT/MY2019/000025

§ 371 Date: Jan. 6, 2021

Related U.S. Application Data
(60) Provisional application No. 62/698,558, filed on Jul. 16, 2018.

(51) Int. Cl. A61K 31/192; A61K 45/06; A61P 25/28; A61M 1/34; A61N 1/18

(52) U.S. Cl. CPC A61K 31/192; A61K 45/06; A61N 1/18; A61M 1/3496; A61P 25/28

(57) ABSTRACT

The present disclosure provides a method of preventing or treating anti-NMDAR encephalitis in a subject in need thereof, including administering to the subject an effective amount of benzoic acid or a salt and derivative thereof.

### BACKGROUND

### 1. Technical Field

The present disclosure relates to prophylaxis or treatment of anti-N-methyl-D-aspartate receptor (NMDAR) encephalitis, and particularly to methods for preventing or treating anti-NMDAR encephalitis by use of benzoates.

### 2. Description of Associated Art

Anti-N-methyl-D-aspartate receptor (NMDAR) encephalitis is an autoimmune encephalitis caused by NMDAR dysfunction associated with autoantibodies. Its occurrence has been increasingly identified in the past decade. The autoantibodies pass the blood-brain barrier and selectively crosslink with NMDA receptors, resulting in internalization of NMDA receptors in both excitatory and inhibitory hippocampal neurons that eventually causes hypofunction of NMDAR-mediated synaptic neurotransmission.

Majority of patients affected with anti-NMDAR encephalitis revealed first the psychotic symptom, followed by neurologic symptom such as epilepsy. Usually, anti-epileptics and neuroleptic drugs are less effective in patients with anti-NMDAR encephalitis. In addition, therapies involving the removal of autoantibodies by immune therapy with methylprednisolone and intravenous immunoglobulin or by plasma exchange are also used to treat patients with anti-NMDAR encephalitis. However, some patients remain refractory even after immune therapy and antipsychotic treatment.

Accordingly, there remains a need for the effective prophylaxis or treatment of anti-NMDAR encephalitis.

### SUMMARY

In view of the foregoing, the present disclosure provides a method of preventing or treating anti-NMDAR encephalitis in a subject in need thereof, comprising administering to the subject an effective amount of benzoic acid or a salt and derivative thereof and a pharmaceutically acceptable excipient.

In one embodiment of the present disclosure, the benzoic acid salt and derivative thereof is sodium benzoate, potassium benzoate, calcium benzoate, magnesium benzoate, 2-aminobenzoate, 3-aminobenzoate, 4-aminobenzoate, ethyl 4-hydroxybenzoate, sodium ethyl 4-hydroxybenzoate, propyl-4-hydroxybenzoate, sodium propyl-4-hydroxybenzoate, methyl 4-hydroxybenzoate, sodium 4-hydroxybenzoate, benzyl benzoate, or methyl benzoate, but not limited thereto.

In another embodiment of the present disclosure, the benzoic acid salt is sodium benzoate.

In one embodiment of the present disclosure, the pharmaceutically acceptable excipient is selected from the group consisting of a filler, a binder, a preservative, a disintegrating agent, a lubricant, a suspending agent, a wetting agent, a solvent, a surfactant, an acid, a flavoring agent, polyethylene glycol (PEG), alkylene glycol, sebacic acid, dimethyl sulfoxide, an alcohol, and any combination thereof.

In one embodiment of the present disclosure, the anti-NMDAR encephalitis is caused by the hypofunction of NMDAR. In another embodiment, the cellular damage in anti-NMDAR encephalitis is caused by autoantibodies.

In one embodiment of the present disclosure, the benzoic acid or the salt and derivative thereof is administered to the subject in an effective amount to enhance or activate NMDAR function, thereby preventing or treating anti-NMDAR encephalitis.

In one embodiment of the present disclosure, the benzoic acid or the salt and derivative thereof may be administered to the subject in an amount ranging from 200 mg/day to 2000 mg/day, such as from 400 mg/day to 1800 mg/day, from 600 mg/day to 1500 mg/day, and from 800 mg/day to 1200 mg/day.

In one embodiment of the present application, the benzoic acid or the salt and derivative thereof may be administered to the subject in a period ranging from 1 month to 2 years, such as from 4 weeks to 12 months. In another embodiment of the present application, the benzoic acid or the salt and derivative thereof is administered to the subject in a period of around 2 months.

In one embodiment of the present disclosure, the benzoic acid or the salt and derivative thereof serves as a sole active ingredient for preventing or treating anti-NMDAR encephalitis in the composition, or the benzoic acid or the salt and derivative thereof is in combination with an additional active ingredient for preventing or treating the anti-NMDAR encephalitis. In one embodiment, the additional active ingredient is an antipsychotics. In another embodiment, the antipsychotics is selected from the group consisting of aripiprazole, clozapine, paliperidone, risperidone, brexpiprazole, olanzapine, quetiapine, ziprasidone, amisulpride, asenapine, iloperidone, lurasidone cariprazine, zotepine, haloperidol, chlorpromazine, clotiapine, flupentixol, clopixol, sulpiride, chlorprothixene, levomepromazine, mesoridazine, periciazine, promazine, thioridazine, loxapine, molindone, perphenazine, thiothixene, droperidol, fluphenazine, pimozide, prochlorperazine, thioproperazine, trifluoperazine, zuclopenthixol.

In one embodiment of the present disclosure, administration of the benzoic acid or the salt and derivative thereof is combined with an additional therapy for preventing or treating anti-NMDAR encephalitis. In one embodiment, the additional therapy includes, but not limited to, psychotherapy, electroconvulsive therapy (ECT) and other brain stimulations such as repetitive transcranial magnetic stimulation (rTMS) and transcranial direct current stimulation (tDCS), immune therapy, plasmaphoresis, pulse therapy with a steroid and any combination thereof. In one embodiment, the pulse therapy with a steroid is pulse therapy with methylprednisolone.

In addition to the above, the present disclosure also provides a use of an effective amount of benzoic acid or a salt and derivative thereof for manufacture of a medicament for preventing or treating anti-N-methyl-D-aspartate receptor (NMDAR) encephalitis in a subject in need thereof. Moreover, the present disclosure further provides an effective amount of benzoic acid or a salt and derivative thereof for use in preventing or treating anti-N-methyl-D-aspartate receptor (NMDAR) encephalitis in a subject in need thereof.

### DETAILED DESCRIPTION OF THE EMBODIMENTS

The following examples are used to exemplify the present disclosure. A person of ordinary skill in the art can understand the other advantages of the present disclosure, based on the disclosure of the present specification. The present disclosure can also be implemented or applied as described in different specific examples.

It is further noted that, as used in this specification, the singular forms “a,” “an,” and “the” include plural referents, unless expressly and unequivocally limited to one referent. The term “or” is used interchangeably with the term “and/or” unless the context clearly indicates otherwise.

Anti-NMDAR encephalitis is an autoimmune disease owing to the passing of autoantibodies across the brain-blood barrier, causing the selective crosslinking and internalization of NMDA receptors in both excitatory and inhibitory hippocampal neurons that result in reduction of NMDAR-mediated synaptic currents and hypofunction of NMDAR.

The present disclosure provides methods of preventing or treating anti-NMDAR encephalitis in a subject in need thereof, comprising administering to the subject an effective amount of benzoic acid or a salt and derivative thereof.

As used herein, the term “treating” or “treatment” refers to administration of an effective amount of benzoic acid or a salt and derivative thereof to a subject in need thereof with the purpose of cure, alleviate, relieve, remedy, ameliorate, or prevent the disease, the symptoms thereof, or the predisposition towards it. Such a subject can be identified by a health care professional based on results from any suitable diagnostic method.

In one embodiment of the present disclosure, the subject to be treated by the method of the present disclosure suffers from anti-NMDAR encephalitis. The subject diagnosed with anti-NMDAR encephalitis may have headache and nausea, presenting first a psychiatric symptom, followed with neurologic dysfunction. In a further embodiment of the present disclosure, the psychiatric symptom includes agitation, cognitive deterioration, repetitive dissociative memory impairment and those associated with schizophrenia. In another embodiment of the present disclosure, the neurologic dysfunction in the subject diagnosed with anti-NMDAR encephalitis includes epilepsy. In another embodiment, the subject diagnosed with anti-NMDAR encephalitis has anti-NMDAR antibodies identified in the subject's cerebrospinal fluid (CSF) and serum. In another embodiment, the cellular damage in anti-NMDAR encephalitis is caused by autoantibodies, which pass the blood-brain barrier to enter the brain to selectively crosslink and internalize NMDAR in both the excitatory and inhibitory hippocampal neurons. In one embodiment, the subject to be treated by the method of the present disclosure suffers from anti-NMDAR encephalitis and can be treated with an additional therapy before, after or at the same time with the method of the present disclosure. In a further embodiment, the additional therapy treated with the method of the present disclosure is psychotherapy, electroconvulsive therapy (ECT), immune therapy, plasmaphoresis, pulse therapy with a steroid and any combination thereof. In yet another embodiment, the subject to be treated by the method of the present disclosure suffers from anti-NMDAR encephalitis and shows poor response, or is refractory to other therapies.

Benzoic acid occurs naturally in many plants and animals. It is therefore a natural constituent of many foods, including milk products (IPCS 1993). Benzoic acid and sodium benzoate are also legal food additives in USA (Joint FAO/WHO Expert Committee on Food Additives. 1965, 1973), Taiwan (Department of Health), and World Health Organization (IPCS 1993), and are widely used in manufacturing fruit jelly, butter, soy-bean sauce, processed meat, etc.

D-amino acid oxidase (DAAO) is a flavoenzyme of peroxisomes existing in the brain, kidney and liver of mammals, which is responsible for degrading D-serine, D-alanine, and other D-amino acids. In the present disclosure, the benzoic acid salt such as sodium benzoate is administered to inhibit DAAO activity and thereby raise synaptic concentrations of D-serine and other D-amino acids. In the present disclosure, the benzoic acid or the salt and derivative thereof is administered to a subject in an effective amount to prevent or treat anti-NMDAR encephalitis through enhancing or activating function of NMDAR. In one embodiment of the present disclosure, the benzoic acid or the salt and derivative thereof enhances NMDA function by inhibiting the DAAO activity in a subject with anti-NMDAR encephalitis.

As used herein, the term “effective amount” refers to a therapeutical amount that is sufficient to result in prevention of the development, recurrence, or onset of anti-NMDAR encephalitis and one or more symptoms thereof, to enhance or improve the prophylactic effect of another therapy, reduce the severity, the duration of the disorder, ameliorate one or more symptoms of the disorder, prevent the advancement of anti-NMDAR encephalitis, and/or enhance or improve the therapeutic effect of another therapy.

In some embodiments of the present disclosure, the effective amount of the benzoic acid or the salt and derivative thereof may range from 200 mg/day to 2000 mg/day. In an embodiment, a lower limit of the dosage may be 200 mg/day, 225 mg/day, 250 mg/day, 275 mg/day, 300 mg/day, 325 mg/day, 350 mg/day, 400 mg/day, 450 mg/day, 500 mg/day, 550 mg/day, 600 mg/day, 700 mg/day, 750 mg/day, or 800 mg/day and an upper limit of the dosage may be 2000 mg/day, 1800 mg/day, 1500 mg/day, 1200 mg/day, 1000 mg/day, 900 mg/day, 800 mg/day, 750 mg/day, 700 mg/day, or 600 mg/day. For example, the dosage of the benzoic acid or the salt and derivative thereof may be 225 mg/day to 2000 mg/day, 250 mg/day to 1800 mg/day, 450 mg/day to 1500 mg/day, 500 mg/day to 1200 mg/day, 500 mg/day to 1000 mg/day, 550 mg/day to 1000 mg/day, 600 mg/day to 800 mg/day, about 1000 mg/day, about 900 mg/day, about 750 mg/day, or about 500 mg/day.

As used herein, when a number or a range is recited, a person having ordinary skill in the art understands that it intends to encompass an appropriate, reasonable range for the particular field related to the disclosure.

By reciting at least 200 mg to 2000 mg, it means that all integer unit amounts within the range are specifically disclosed as part of the disclosure. Thus, 200, 201, 202 . . . 250, 251, 252 . . . 1000, 1001, 1002 . . . 1997, 1998, 1999 and 2000 unit amounts are included as embodiments of the present disclosure.

In some embodiments of the present disclosure, the administration of the benzoic acid or the salt and derivative thereof may be conducted, for example, once per day, twice per day, 3 times per day, or 4 times per day. In an embodiment, the administration of the benzoic acid or the salt and derivative thereof may be conducted once per day.

In some embodiments of the present disclosure, the benzoic acid or the salt and derivative thereof may be administered to the subject in a period sufficient to prevent or treat anti-NMDAR encephalitis. The sufficient period may depend on the species, gender, body weight or age of the subject, the stage, symptom or severity of the disease, and the routes, timing or frequency of the administration. In some embodiments of the present disclosure, the administration of the benzoic acid or the salt and derivative thereof is daily over at least 1 month. For example, the period of administration of the benzoic acid or the salt and derivative thereof may last for 1, 2, 3, 4, or 6 months, or 1, 2, 3 or 4 years, or even longer, as long as no side effect occurs during the treatment period. In the exemplary embodiments of the present disclosure, the period may be in a range of from 1 month to 2 years. In another embodiment, the period ranges from 4 weeks to 12 months. In yet another embodiment, the administration of the benzoic acid or the salt and derivative thereof is daily for 2 months.

In one embodiment of the present disclosure, the method involves the use of benzoic acid, benzoic acid salt, or a derivative thereof, which can be selected from the group consisting of benzoic acid, sodium benzoate, potassium benzoate, calcium benzoate, magnesium benzoate, 2-aminobenzoate, 3-aminobenzoate, 4-aminobenzoate, ethyl 4-hydroxybenzoate, sodium ethyl 4-hydroxybenzoate, propyl-4-hydroxybenzoate, sodium propyl-4-hydroxybenzoate, methyl 4-hydroxybenzoate, sodium 4-hydroxybenzoate, benzyl benzoate, and methyl benzoate.

In one embodiment of the present disclosure, the benzoic acid or the salt and derivative thereof is administered in an oral dosage form. In one embodiment of the present disclosure, the benzoic acid or the salt and derivative thereof administered to the subject may be contained in a pharmaceutical composition. The pharmaceutical composition of the present disclosure comprises benzoic acid or a salt and derivative thereof and a pharmaceutically acceptable excipient thereof. In an embodiment, the pharmaceutical composition of the present disclosure is formulated in a form suitable for oral administration, and thus the pharmaceutical composition may be administered to the subject by oral delivery. Alternatively, the pharmaceutical composition may be formulated in a form of dry powder, a tablet, a lozenge, a capsule, granule, or a pill. The pharmaceutically acceptable excipient includes, but is not limited to, a filler, a binder, a preservative, a disintegrating agent, a lubricant, a suspending agent, a wetting agent, a solvent, a surfactant, an acid, a flavoring agent, polyethylene glycol (PEG), alkylene glycol, sebacic acid, dimethyl sulfoxide, an alcohol, or any combination thereof.

The pharmaceutical composition of the present disclosure may only comprise the benzoic acid or the salt and derivative thereof as an active ingredient for preventing or treating anti-NMDAR encephalitis. In other words, the benzoic acid or the salt and derivative thereof serves as the only active ingredient for preventing or treating anti-NMDAR encephalitis in the pharmaceutical composition of the present disclosure. In this embodiment, the present disclosure provides a safe and effective therapy for preventing or treating anti-NMDAR encephalitis by the use of the benzoic acid or the salt and derivative thereof alone as the active ingredient.

Alternatively, in another embodiment, the pharmaceutical composition may be administered to a subject in combination with another active ingredient, unless the effect of the disclosure is inhibited. The benzoic acid or the salt and derivative thereof and another active ingredient may be provided in a single composition or in separate compositions.

In an embodiment, the administration of the benzoic acid or the salt and derivative thereof in the method provided by the present disclosure may be combined with any suitable conventional therapy for anti-NMDAR encephalitis. In an embodiment, the conventional therapy for anti-NMDAR encephalitis includes, but is not limited to, the first or second line immune therapy, pulse therapy with a steroid, electroconvulsive therapy and antipsychotics therapy.

Many examples have been used to illustrate the present disclosure. The examples below should not be taken as any limit to the scope of the present disclosure.

### EXAMPLE

The present disclosure examined the efficacy and safety of sodium benzoate for the treatment of anti-NMDAR encephalitis.

A previously healthy 17-year-old, male presented with headache and nausea without fever. One week later, he progressively developed agitation, cognitive deterioration with required psychiatric evaluation. The patient was admitted to acute psychiatric ward 3 times due to suspected schizophrenia and repetitive dissociative memory impairment.

The patient was treated with a heavy dose of antipsychotics and electroconvulsive therapy (ECT) but showed very poor response to antipsychotics even at high doses (aripiprazole 20-30 mg, clozapine 300-500 mg, and clozapine 500 mg plus paliperidone 9 mg), and electroconvulsive therapy (thrice a week for three weeks). Anti-NMDAR antibodies were then identified in cerebrospinal fluid (CSF) and serum, and anti-NMDAR encephalitis was confirmed. Brain magnetic resonance imaging was found normal.

The patient was immediately treated with intravenous immunoglobulin (IVIg) and plasmaphoresis after pulse therapy with methylprednisolone. However, no remarkable clinical improvement was noticed after two months of immunotherapy and antipsychotic treatment.

Sodium benzoate was then administered to the patient at a dose of 1000 mg per day for 2 months. The psychotic symptoms including dissociation and agitation were reduced. An obvious clinical improvement was observed on the patient, and he was able to return to a normal life.

The foregoing descriptions of the detailed embodiments are only illustrated to disclose the principle and functions of the present disclosure and do not intend to restrict the scope of the present disclosure. It should be understood to those skilled in the art that all modifications and variations according to the spirit and principle of the present disclosure should fall within the scope of the appended claims. It is intended that the specification and examples are considered as exemplary only, with a full scope of the disclosure being indicated by the following claims. The references listed below in the application are each incorporated by reference as if they were incorporated individually.

DALMAU, J. (2016) NMDA receptor encephalitis and other antibody-mediated disorders of the synapse: The 2016 Cotzias Lecture. Neurology, 87, 2471-2482.

DALMAU, J., LANCASTER, E., MARTINEZ-HERNANDEZ, E., ROSENFELD, M. R. & BALICE-GORDON, R. (2011) Clinical experience and laboratory investigations in patients with anti-NMDAR encephalitis. Lancet Neurol., 10, 63-74.

LEYPOLDT, F., ARMANGUE, T. & DALMAU, J. (2015) Autoimmune encephalopathies. Annals of the New York Academy of Sciences, 1338, 94-114.

TITULAER, M. J., MCCRACKEN, L., GABILONDO, I., ARMANGUE, T., GLASER, C., IIZUKA, T., HONIG, L. S., BENSELER, S. M., KAWACHI, I., MARTINEZ-HERNANDEZ, E., AGUILAR, E., GRESA-ARRIBAS, N., RYAN-FLORANCE, N., TORRENTS, A., SAIZ, A., ROSENFELD, M. R., BALICE-GORDON, R., GRAUS, F. & DALMAU, J. (2013) Treatment and prognostic factors for long-term outcome in patients with anti-NMDA receptor encephalitis: an observational cohort study. Lancet Neurol., 12, 157-165.

TSUTSUI, K., KANBAYASHI, T., TAKAKI, M., OMORI, Y., IMAI, Y., NISHINO, S., TANAKA, K. & SHIMIZU, T. (2017) N-methyl-D-aspartate receptor antibody could be a cause of catatonic symptoms in psychiatric patients: case reports and methods for detection. Neuropsychiatr. Dis. Treat., 13, 339-345.

1.-15. (canceled)

16. A method for preventing or treating anti-N-methyl-D-aspartate receptor (NMDAR) encephalitis in a subject in need thereof, comprising administering to the subject an effective amount of a benzoic acid salt or a benzoic acid derivative in combination with an immune therapy for preventing or treating anti-NMDAR encephalitis.

17. The method according to claim 16, wherein the benzoic acid salt is sodium benzoate, potassium benzoate, calcium benzoate, magnesium benzoate, 2-aminobenzoate, 3-aminobenzoate, 4-aminobenzoate, sodium ethyl 4-hydroxybenzoate, sodium propyl-4-hydroxybenzoate, or sodium 4-hydroxybenzoate, and the benzoic acid derivative is ethyl 4-hydroxybenzoate, propyl-4-hydroxybenzoate, methyl 4-hydroxybenzoate, benzyl benzoate, or methyl benzoate.

18. The method according to claim 16, wherein the benzoic acid salt is sodium benzoate.

19. The method according to claim 16, wherein the anti-NMDAR encephalitis is caused by hypofunction of NMDAR.

20. The method according to claim 16, wherein the administering the benzoic acid salt or the benzoic acid derivative enhances or activates NMDAR function in the subject.

21. The method according to claim 16, wherein the benzoic acid salt or the benzoic acid derivative is administered to the subject in an amount ranging from 200 mg/day to 2000 mg/day.

22. The method according to claim 16, wherein the benzoic acid salt or the benzoic acid derivative is administered to the subject in an amount ranging from 400 mg/day to 1800 mg/day.

23. The method according to claim 16, wherein the benzoic acid salt or the benzoic acid derivative is administered to the subject in an amount ranging from 450 mg/day to 1500 mg/day.

24. The method according to claim 16, wherein the benzoic acid salt or the benzoic acid derivative is administered to the subject in an amount ranging from 500 mg/day to 1000 mg/day.

25. The method according to claim 16, wherein the benzoic acid salt or the benzoic acid derivative is administered to the subject in a period ranging from 1 month to 2 years.

26. The method according to claim 16, wherein the benzoic acid salt or the benzoic acid derivative is administered to the subject in a period ranging from 4 weeks to 12 months.

27. The method according to claim 16, wherein the benzoic acid salt or the benzoic acid derivative is administered to the subject in combination with an additional active ingredient for preventing or treating the anti-NMDAR encephalitis.

28. The method according to claim 27, wherein the additional active ingredient is an antipsychotic.

29. The method according to claim 16, wherein the benzoic acid salt or the benzoic acid derivative is administered to the subject in combination with an additional therapy for preventing or treating anti-NMDAR encephalitis.

30. The method according to claim 29, wherein the additional therapy includes psychotherapy, electroconvulsive therapy (ECT), plasmaphoresis, pulse therapy with a steroid, or any combination thereof.
